(19) United States
(12) Patent Application Publication
(10) Pub. No.: US 2026/0182985 A1
(43) Pub. Date: Jul. 2, 2026

(54) BIOELECTRONIC SURGICAL SUTURE FOR MONITORING SUTURE TENSION

(71) Applicant: Zhejiang University, Hangzhou (CN)

(72) Inventors: Kaichen Xu, Hangzhou (CN); Yuhong Xu, Hangzhou (CN); Tianyu Li, Hangzhou (CN); Yifan Wang, Hangzhou (CN); Xiujun Cai, Hangzhou (CN); Huayong Yang, Hangzhou (CN)

(21) Appl. No.: 19/432,913

(22) Filed: Dec. 24, 2025

(30) Foreign Application Priority Data
Dec. 27, 2024 (CN) 202411950857.X

(51) Int. Cl. A61B 17/06; A61B 17/00; A61B 17/04; A61B 90/00; A61L 17/14

(52) U.S. Cl. CPC A61B 17/06166; A61L 17/14; A61B 2017/0003; A61B 2017/0496; A61B 2090/064

(57) ABSTRACT

A bioelectronic surgical suture for monitoring suture tension includes a conductive material made of electropositive material, and a sleeve made of electronegative material. The sleeve is a hollow capillary, the conductive material is arranged in the sleeve through infusion, and is processed by a first laser scanning operation after infusion. An outer wall of the sleeve has a micro-nano structure manufactured by a second laser scanning operation.

### CROSS-REFERENCE TO RELATED APPLICATION

This application claims priority of Chinese Patent Application No. 202411950857.X, filed on Dec. 27, 2024, the entire contents of which are incorporated herein by reference.

### TECHNICAL FIELD

The present disclosure belongs to the field of bioelectronics and flexible sensing technology, and particularly relates to a bioelectronic surgical suture for monitoring suture tension.

### BACKGROUND

The suture process in current clinical surgery mainly relies on the doctor's surgical experience, especially in terms of controlling the tightness of suture. There is usually no standardized or quantitative measurement method. Doctors need to judge the tension and tightness of the suture through visual inspection and hand feeling when suturing. This method is not only greatly influenced by personal experience, but also the definition of appropriate tightness may differ greatly among different doctors. Due to the lack of an accurate quantitative feedback mechanism, the tightness of sutures is often difficult to ensure consistency and accuracy.

Improper suture tightness can cause a series of postoperative complications. For example, overly tight suturing may lead to local tissue ischemia and necrosis, affecting the wound healing process; whereas overly loose suturing may lead to unstable wound edges, thereby increasing the risk of wound dehiscence. These problems may not only prolong the patient's recovery time, but also lead to complications, seriously affecting the postoperative efficacy, and even requiring secondary surgery for repair.

### SUMMARY

The present disclosure relates to a bioelectronic surgical suture, and in particular to a bioelectronic surgical suture capable of monitoring suturing force and knotting force in real time during suturing and knotting processes. With the advancement of modern medical technology, surgical accuracy and postoperative recovery have become important indicators of clinical surgery. In order to improve surgical effects and reduce postoperative complications, the present disclosure innovatively provides a bioelectronic surgical suture for monitoring suture tension. The surgical suture can monitor suturing force and knotting force in real time, and can effectively assist doctors in performing more accurate surgical operations, thereby optimizing suture effects, and reducing postoperative complications and promoting wound healing.

In order to achieve the above purpose, the present disclosure adopts the following technical solutions.

A bioelectronic surgical suture for monitoring suture tension includes: a conductive material made of electropositive material, and a sleeve made of electronegative material. The sleeve is a hollow capillary, the conductive material is arranged in the sleeve through infusion, and is processed by a first laser scanning operation after infusion. An outer wall of the sleeve has a micro-nano structure manufactured by a second laser scanning operation. The bioelectronic surgical suture is a self-powered sensor, and can monitor the suture tension on the bioelectronic surgical suture.

In the above technical solutions, the conductive material is selected from at least one of biocompatible conductive polymers (such as ionic conductive hydrogels, electronic conductive hydrogels, etc.), poly (3,4-ethylenedioxythiophene)-polystyrene sulfonate (PEDOT:PSS), ionic liquid mixed PEDOT:PSS (IL/PEDOT:PSS), carbon materials (such as carbon nanotubes etc.), metallic materials (such as liquid metals, silver paste, gold nanoparticles, silver nanowires, etc.).

The sleeve is a biocompatible polymer selected from at least one of polytetrafluoroethylene (PTFE), polydimethylsiloxane (PDMS), silicone, or polyurethane (TPU). The selected sleeve is made of a transparent material with low light absorption characteristics, which is more conducive to subsequent laser processing.

Further, an outer diameter size of the sleeve corresponds to an existing surgical suture used in clinical applications, including but not limited to surgical suture models: 3-0, 2-0, 0, 1, 2, 3, and 4. Two ends of the bioelectronic surgical suture are arranged with a suture needle and a wire; and the suture needle is connected to the sleeve for penetrating the sutured tissue, and the wire is connected to the conductive material for connecting signal acquisition equipment.

Further, the first laser scanning operation includes: scanning the conductive material along the sleeve with a first laser, rotating the sleeve to change an angle of the sleeve, and scanning the conductive material again. In the first laser scanning operation, the first laser is a continuous-wave laser (CW laser) with a wavelength of 532 nm, a laser power control range of 0.02-0.1 W, a speed control range of 50-200 mm/s, and a line spacing control range of 0.01-0.05 mm. For example, a CW laser is used to scan a sleeve infused with a conductive material, and the electromechanical properties of the inner conductive material are improved through the thermal field and electric field provided by the CW laser. The sleeve is a transparent material with low light absorption characteristics and low absorption in this laser band, so the laser will not be significantly absorbed by the sleeve, but will mostly penetrate the sleeve or directly act on the conductive material inside the sleeve.

Further, the second laser scanning operation includes: scanning the conductive material along the sleeve with a second laser, rotating the sleeve to change an angle of the sleeve, and scanning the conductive material again. The second laser is a carbon dioxide infrared laser, a femtosecond infrared laser, or a femtosecond ultraviolet laser. The laser thermal effect is controlled by controlling the parameters of the second laser, so that the second laser only acts on the sleeve. The commonly used laser frequency range is 50-100 KHz, the laser speed is 50 -250 mm/s, the line spacing is 0.03-0.2 mm, and the pulse width is 2-10 μs. For example, femtosecond laser is used to scan the sleeve after the first laser scanning treatment along the axis, and micro-nano structures are processed on the outer wall of the outer sleeve through the rapid thermal effect provided by the femtosecond laser. Due to the low thermal effect of femtosecond laser, the impact of the pulse energy used for surface micro-nano structure processing on the conductive material in the outer sleeve can be negligible.

The surgical suture is based on the principle of triboelectric nanogenerator, the suture tension of the bioelectronic surgical suture causes changes in a contact area between the bioelectronic surgical suture and a sutured tissue, and output electrical signals changes to monitor the suture tension on the bioelectronic surgical suture. It generates current through charge transfer with sutured tissue and does not require additional power supply support. It is a self-powered sensor. The bioelectronic surgical suture is capable of providing different quantitative feedback according to different suture methods (including but not limited to intermittent suture, continuous suture, and purse-string suture), different suture sites (including but not limited to the surface of the skin, deep intestines, and internal organs), or different knotting methods (single knot, square knot, and surgical knot). The surgical suture can also combine the doctor's clinical suture experience to assist in establishing a tension quantitative model, thereby helping the doctor optimize surgical operations, reducing postoperative complications, and promoting wound healing.

The beneficial effects of the present disclosure are provided as follows.

The disclosed bioelectronic surgical suture can monitor changes in suturing force and knotting force in real time during the operation, generate dynamic feedback signals, help doctors adjust operations based on real-time data, thereby ensuring accurate control of suture force, and optimizing suture effect. Through real-time monitoring of suturing force and knotting force, the incidence of postoperative complications can be effectively reduced and wound healing can be promoted.

The disclosed bioelectronic surgical suture brings a new perspective to suture technology, expands the potential application of laser manufacturing technology in the field of flexible electronics, and provides new insights for interdisciplinary research on bioelectronic devices in medical clinical diagnosis.

### BRIEF DESCRIPTION OF THE DRAWINGS

The accompanying drawings are used to provide a further understanding of the present disclosure and form a part of the specification. Together with the embodiments of the present disclosure, the drawings are used to explain the present disclosure and do not constitute a limitation of the present disclosure. In the accompanying drawings:

FIG. 1 is a schematic diagram of the structure and application of a bioelectronic surgical suture in the present disclosure;

FIG. 2A is a schematic diagram of the physical object of the bioelectronic surgical suture involved in the present disclosure;

FIG. 2B is a schematic diagram of the microstructure of the bioelectronic surgical line involved in the present disclosure;

FIG. 3 is a schematic diagram of a preparation flow of inner conductive material in an embodiment of the present disclosure;

FIG. 4A is a schematic diagram of inner conductive material before preparation in an embodiment of the present disclosure;

FIG. 4B is a schematic diagram of inner conductive material after preparation in an embodiment of the present disclosure;

FIG. 5A is a schematic diagram of an original conductive material acting on a planar film in an embodiment of the present disclosure;

FIG. 5B is a schematic diagram of the original conductive material mixed with ionic liquid acting on a planar film in an embodiment of the present disclosure;

FIG. 5C is a schematic diagram of the processing of a mixed conductive material acting on a planar film through laser action in an embodiment of the present disclosure;

FIG. 6 is a schematic diagram of the comparison of electrical properties of different conductive material films before and after CW laser action in an embodiment of the present disclosure;

FIG. 7 is a schematic diagram of the comparison of electrical properties of a conductive material film under different CW laser parameters in an embodiment of the present disclosure;

FIG. 8A is a schematic diagram of mechanical properties of different conductive material films before CW laser action in an embodiment of the present disclosure;

FIG. 8B is a schematic diagram of mechanical properties of different conductive material films after CW laser action in an embodiment of the present disclosure;

FIG. 9 is a schematic diagram of a manufacturing process of the bioelectronic surgical suture involved in the present disclosure;

FIG. 10 is a schematic diagram of the bioelectronic surgical suture involved in the present disclosure under the action of CW laser;

FIG. 11A is a schematic diagram of the knotting of a bioelectronic surgical suture without surface micro-nano structures involved in the present disclosure;

FIG. 11B is a schematic diagram of the knotting of a bioelectronic surgical suture with surface micro-nano structures involved in the present disclosure;

FIG. 12 is a schematic diagram of the bioelectronic surgical suture involved in the present disclosure when it is sutured to tissue;

FIG. 13A is a schematic diagram of an output signal of the bioelectronic surgical suture without surface micro-nano structure involved in the present disclosure;

FIG. 13B is a schematic diagram of an output signal of the bioelectronic surgical suture with surface micro-nano structure involved in the present disclosure;

FIG. 14A is a schematic diagram of a surface micro-nano structure of an outer sleeve under a speed parameter of 50 mm/s of femtosecond laser;

FIG. 14B is a schematic diagram of the surface micro-nano structure of the outer sleeve under a speed parameter of 100 mm/s of femtosecond laser;

FIG. 14C is a schematic diagram of the surface micro-nano structure of the outer sleeve under a speed parameter of 150 mm/s of femtosecond laser;

FIG. 14D is a schematic diagram of the surface micro-nano structure of the outer sleeve under a speed parameter of 200 mm/s of femtosecond laser;

FIG. 14E is a schematic diagram of the surface micro-nano structure of the outer sleeve under a speed parameter of 250 mm/s of femtosecond laser;

FIG. 15A is a schematic diagram of the surface micro-nano structure of the outer sleeve under a line spacing of 0.03 mm of femtosecond laser;

FIG. 15B is a schematic diagram of the surface micro-nano structure of the outer sleeve under a line spacing of 0.05 mm of femtosecond laser;

FIG. 15C is a schematic diagram of the surface micro-nano structure of the outer sleeve under a line spacing of 0.15 mm of femtosecond laser;

FIG. 15D is a schematic diagram of the surface micro-nano structure of the outer sleeve under a line spacing of 0.20 mm of femtosecond laser;

FIG. 16A is a schematic diagram of the surface micro-nano structure of the outer sleeve under a pulse width of 8 μs of femtosecond laser;

FIG. 16B is a schematic diagram of the surface micro-nano structure of the outer sleeve under a pulse width of 6 μs of femtosecond laser;

FIG. 16C is a schematic diagram of the surface micro-nano structure of the outer sleeve under a pulse width of 4 μs of femtosecond laser;

FIG. 16D is a schematic diagram of the surface micro-nano structure of the outer sleeve under a pulse width of 2 μs of femtosecond laser;

FIG. 17 is a schematic diagram of the comparison of output signals generated by the contact between the bioelectronic surgical suture and the sutured tissue involved in the present disclosure under different speed parameters of femtosecond laser;

FIG. 18A is a schematic diagram of the comparison of output signals generated by the contact between the bioelectronic surgical suture and the sutured tissue involved in the present disclosure under different line spacing parameters of femtosecond laser;

FIG. 18B is a schematic diagram of the comparison of output signals generated by the contact between the bioelectronic surgical suture and the sutured tissue involved in the present disclosure under different pulse width parameters of femtosecond laser;

FIG. 19A is a line friction stage in which the bioelectronic surgical suture and the sutured tissue involved in the present disclosure change with the change of tension;

FIG. 19B is a knot friction stage in which the bioelectronic surgical suture and the sutured tissue involved in the present disclosure change with the change of tension;

FIG. 20A is a schematic diagram of the signal output by the bioelectronic surgical suture involved in the present disclosure as the tension increases in stages;

FIG. 20B is a schematic diagram of the signal output by the bioelectronic surgical suture in the present disclosure after repeated tightening and loosening with different tensions applied the surgical suture;

FIG. 21 is a schematic diagram of the bioelectronic surgical suture involved in the present disclosure being sutured by purse-string suture;

FIG. 22 is a schematic diagram of a signal of the bioelectronic surgical suture, which changes with the change of tension under purse-string suture;

FIG. 23 is a schematic diagram of real-time monitoring signals during the entire stage of purse-string suture;

FIG. 24A is a schematic diagram of different knotting methods of the bioelectronic surgical suture involved in the present disclosure;

FIG. 24B is a schematic diagram of signal changes in different knotting methods of the bioelectronic surgical suture involved in the present disclosure;

FIG. 25 is a schematic diagram of biocompatibility results of materials involved in embodiments of the present disclosure;

FIG. 26A is a schematic diagram of the bioelectronic surgical suture involved in the present disclosure for suturing a large intestine with different suture tightness;

FIG. 26B is a schematic diagram of the bioelectronic surgical suture involved in the present disclosure for suturing a small intestine with different suture tightness;

FIG. 27A is a schematic diagram of the bioelectronic surgical suture involved in the present disclosure for suturing a tissue in the abdomen of a living rabbit in a normal state;

FIG. 27B is a schematic diagram of output electrical signals during two suture stages of suturing the abdomen of a living rabbit in a normal state using the bioelectronic surgical suture involved in the present disclosure;

FIG. 27C is a schematic diagram of a healing wound 7 days after suturing the abdomen of a living rabbit in a normal state by using the bioelectronic surgical suture involved in the present disclosure;

FIG. 27D is a schematic cross-sectional view of a wound area 7 days after suturing the abdomen of a living rabbit in a normal state by using the bioelectronic surgical suture involved in the present disclosure;

FIG. 28A is a schematic diagram of the bioelectronic surgical suture involved in the present disclosure for suturing a tissue in the abdomen of a living rabbit in an overly loose state;

FIG. 28B is a schematic diagram of output electrical signals during two suture stages of suturing the abdomen of a living rabbit in an overly loose state using the bioelectronic surgical suture involved in the present disclosure;

FIG. 28C is a schematic diagram of a healing wound 7 days after suturing the abdomen of a living rabbit in an overly loose state by using the bioelectronic surgical suture involved in the present disclosure;

FIG. 28D is a schematic cross-sectional view of a wound area 7 days after suturing the abdomen of a living rabbit in an overly loose state by using the bioelectronic surgical suture involved in the present disclosure;

FIG. 29A is a schematic diagram of the bioelectronic surgical suture involved in the present disclosure for suturing a tissue in the abdomen of a living rabbit in an overly tight state;

FIG. 29B is a schematic diagram of output electrical signals during two suture stages of suturing the abdomen of a living rabbit in an overly tight state using the bioelectronic surgical suture involved in the present disclosure;

FIG. 29C is a schematic diagram of a healing wound 7 days after suturing the abdomen of a living rabbit in an overly tight state by using the bioelectronic surgical suture involved in the present disclosure; and

FIG. 29D is a schematic cross-sectional view of a wound area 7 days after suturing the abdomen of a living rabbit in an overly tight state by using the bioelectronic surgical suture involved in the present disclosure.

### DETAILED DESCRIPTION

The technical solutions in embodiments of the present disclosure will be clearly and completely described below in conjunction with the drawings in embodiments of the present disclosure. Obviously, the described embodiments are only a part of the embodiments of the present disclosure, not all embodiments. Based on the embodiments in this disclosure, all other embodiments obtained by a person of ordinary skill in the art without creative effort fall within the scope of protection of this disclosure.

In view of the problems existing in related technologies, developing a surgical suture that can monitor suture tension in real time and provide feedback on suture strength in vitro and in situ is of great significance for improving the accuracy and consistency of surgery. This surgical suture with real-time monitoring function can provide objective quantitative data during the suture process, helping doctors accurately adjust the tightness of the suture and ensuring the stability of the suture and appropriate tissue pressure. In addition, through continuous monitoring of postoperative wound tension, the surgical suture can also help medical staff detect potential abnormalities in a timely manner during the postoperative stage, so as to take effective intervention measures to maximize the patient's rehabilitation process and reduce complications, improving the surgical quality and the overall treatment effect of the patient. FIG. 1 is a schematic diagram of the structure and application of a bioelectronic surgical suture in the present disclosure.

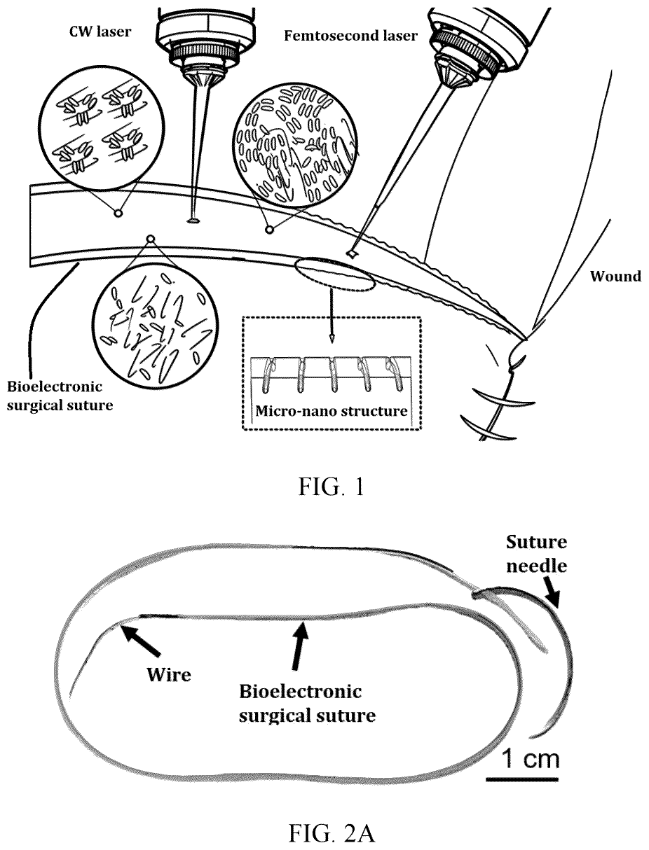

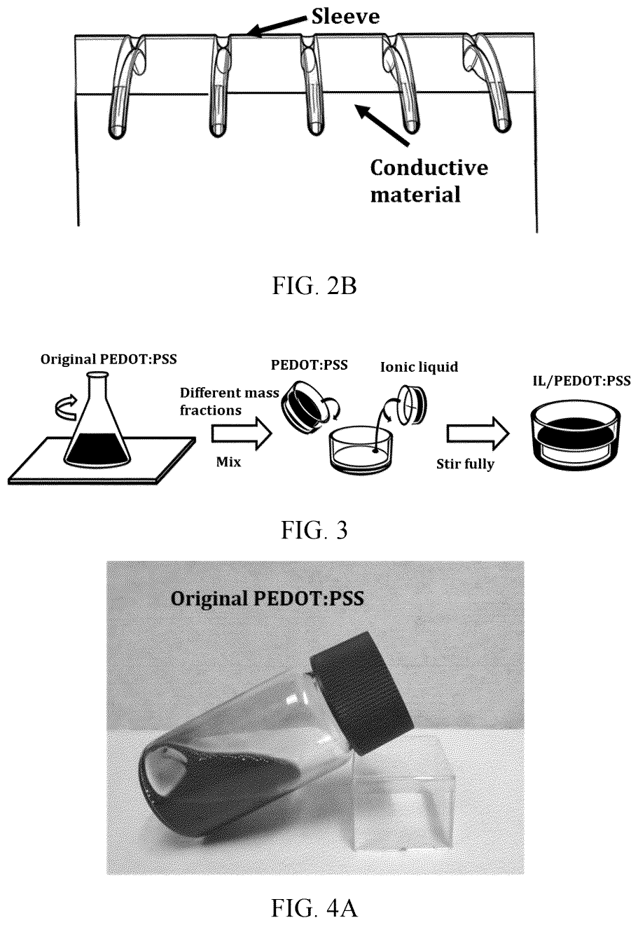

Referring to FIGS. 2A and 2B, the present disclosure provides a technical solution: a bioelectronic surgical suture for monitoring suture tension includes an outer sleeve and an inner conductive material, and two ends of the surgical suture are arranged with a suture needle and a wire (i.e., the suture needle is located on one end, and the wire is located on the other end). The suture is connected to the sleeve and the wire is connected to the conductive material.

The outer sleeve is a hollow capillary made of electronegative materials, including but not limited to biocompatible PTFE, PDMS, silicone, TPU, etc.

The conductive material is an electropositive material, including but not limited to biocompatible conductive polymers, conductive hydrogels, carbon materials, or metallic materials, such as ionic conductive hydrogels, electronic conductive hydrogels, PEDOT:PSS, IL/PEDOT:PSS, carbon nanotubes, liquid metals, silver paste, gold nanoparticles, and silver nanowires.

Preferably, for the bioelectronic surgical suture, the outer diameter size of the outer sleeve corresponds to the actual surgical suture (i.e., existing surgical sutures), including but not limited to surgical suture models: 3-0, 2-0, 0, 1, 2, 3, and 4.

Further, the conductive material is usually filled into the outer sleeve in a flowing state. In order to improve the electromechanical properties of the conductive material and improve the monitoring effect of the bioelectronic surgical suture, the conductive material is modified by laser technology.

Further, the outer sleeve is usually a sleeve with smooth inside and outside. In order to improve the roughness of the outer wall and increase the friction between materials while increasing the specific surface area, the outer wall is processed by laser technology.

In the embodiments described in this disclosure, the outer sleeve is a PTFE capillary, the inner conductive material is a mixed conductive material of ionic liquid mixed with PEDOT:PSS, the laser for modifying the conductive material is CW laser, and the laser for processing surface micro-nano structures is femtosecond laser.

In the embodiments described in this disclosure, as an alternative implementation, this embodiment selected a PEDOT:PSS solution with a model of PH 1000, with a solid phase content of 1.3 wt %, and a mass ratio of PEDOT to PSS of 2:5. The PEDOT:PSS solution was filtered through a 0.45 μm needle type water microporous membrane to a container for later use.

In the embodiments described in this disclosure, as an alternative implementation, the ionic liquid can be a carboxylic acid choline ionic liquid, a methanesulfonate ionic liquid or other carboxyl-containing ionic liquids, which has/have good biocompatibility. Methanesulfonate ionic liquid was selected in this embodiment.

Referring to FIG. 3, in order to achieve the above purpose, the inner conductive material was prepared. PEDOT:PSS is an excellent biocompatible conductive polymer, but in order to improve the electromechanical properties of the original PEDOT:PSS, biocompatible ionic liquids with different mass fractions were stirred and mixed with the original PEDOT:PSS to form a mixed material IL/PEDOT:PSS, and the performance was initially improved by phase separation.

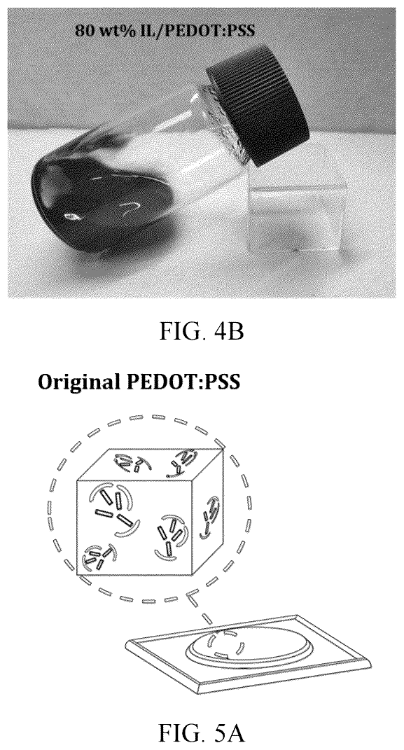

Referring to FIGS. 4A and 4B, the mixed IL/PEDOT:PSS material was more viscous than the original PEDOT:PSS.

Further, in order to demonstrate the improving effect of IL/PEDOT:PSS material, the thin film formed by the original PEDOT:PSS on the substrate was compared with the thin film formed by the IL/PEDOT:PSS material on the substrate.

In the embodiments described in this disclosure, the mass fraction of the ionic liquid relative to the PEDOT:PSS solution was 40-80 wt %. The resulting mixed solution was stirred to form a uniform solution, and the stirring method used ultrasonic stirring for 10-30 minutes. The uniform solution was transferred to a planar substrate by a spin coating method at a spin coating speed of 1500-5000 rpm (revolutions per minute). After spin coating, the planar substrate was placed in a vacuum environment for preliminary drying at 60-80° C. for 15-30 minutes.

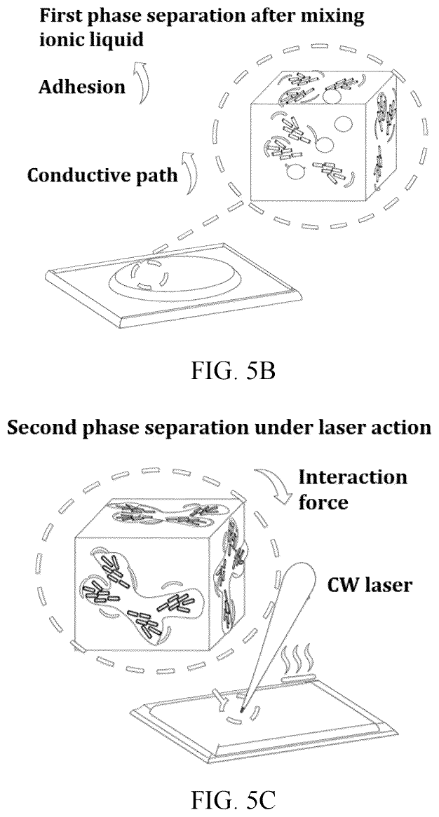

Referring to FIGS. 5A, 5B and 5C, in order to achieve the above purpose, CW laser scanning was performed on the mixed material film, and secondary phase separation was performed through the thermal field and electric field of the laser to improve performance.

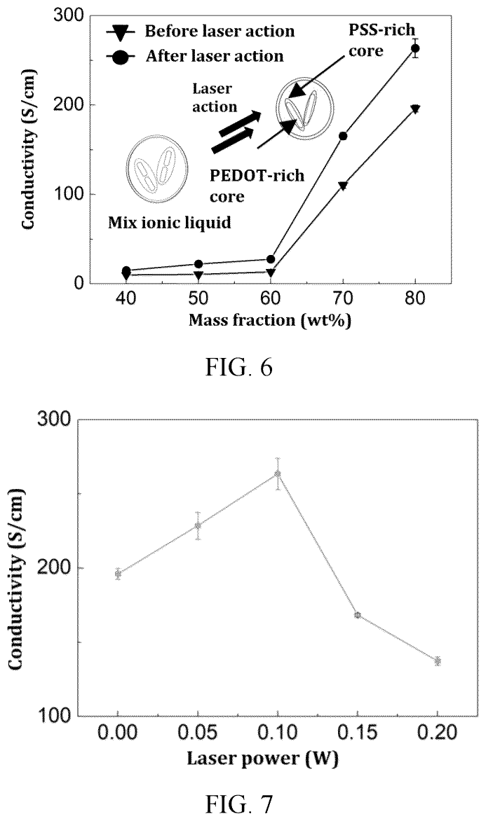

Referring to FIG. 6, comparing the conductivity of mixed material films with different mass fractions of the ionic liquids before and after laser scanning. It was concluded that the optimal mass fraction of ionic liquid relative to PEDOT:PSS solution is 80 wt %.

Referring to FIG. 7, comparing the conductivity of the mixed conductive material film under different CW laser powers, it was concluded that a CW laser power of 0.1 W is the optimal. High power easily causes material carbonization. The conductivity of the mixed conductive material film is 270 S/cm.

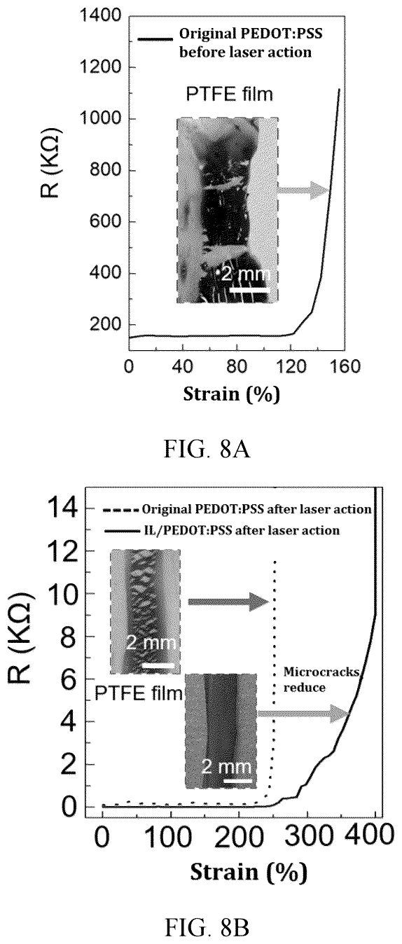

Referring to FIGS. 8A and 8B, comparing the mechanical properties of different conductive material films before and after CW laser action, the elongation of the original conductive material film is 120%, and the elongation of the mixed conductive material film under laser is 400%.

According to the above embodiments, the mixed conductive material was modified under CW laser action.

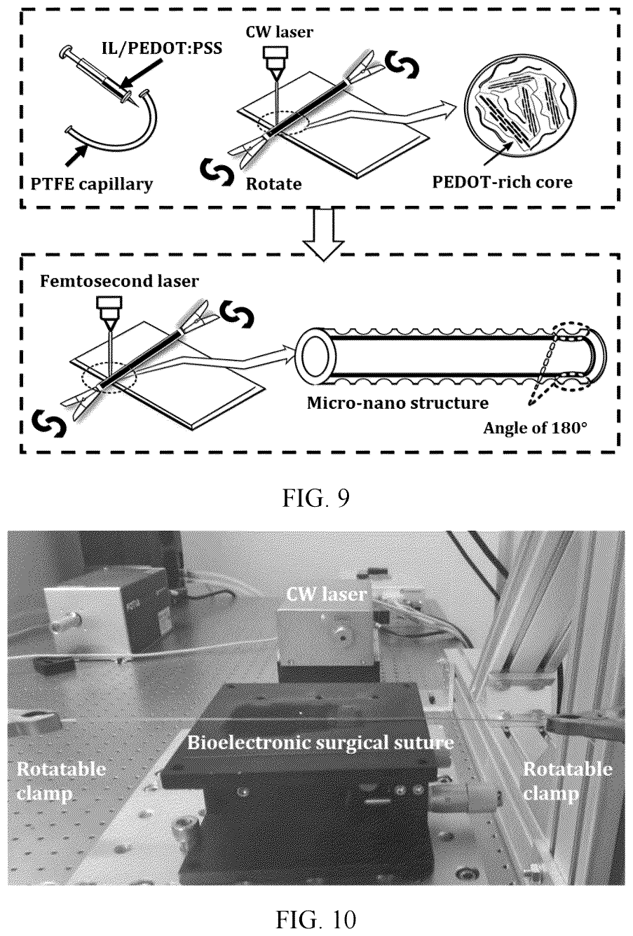

Referring to FIGS. 9-10, the manufacturing process of the bioelectronic surgical suture involved in the embodiments described in this disclosure is described in detail:
In step 1: the mixed conductive solution (IL/PEDOT:PSS) is infused into the PTFE capillary.
In step 2: the two ends of the PTFE capillary are fixed by rotatable clamps, placed in a CW laser environment, and the inner mixed conductive material is modified through CW laser.
In step 3: in a CW laser environment, the laser power is 0.1 W, the speed is 100 mm/s, the line spacing is 0.03 mm, the clamps are rotated at an angle of 180°, and the inner mixed conductive material is fully modify.
In step 4: the PTFE capillary is placed in a femtosecond laser environment, and micro-nano structures are processed on the outer wall of the PTFE capillary through femtosecond laser.
In step 5: the PTFE capillary is placed in a femtosecond laser environment, with a laser frequency of 100 KHz, a speed of 50 -250 mm/s, a line spacing of 0.03-0.2 mm, and a pulse width of 2-10 μs. The clamps are rotated at an angle of 180° to form micro-nano structures on two sides of the capillary (i.e., upper and lower sides of the outer wall in longitudinal direction, see FIG. 9).

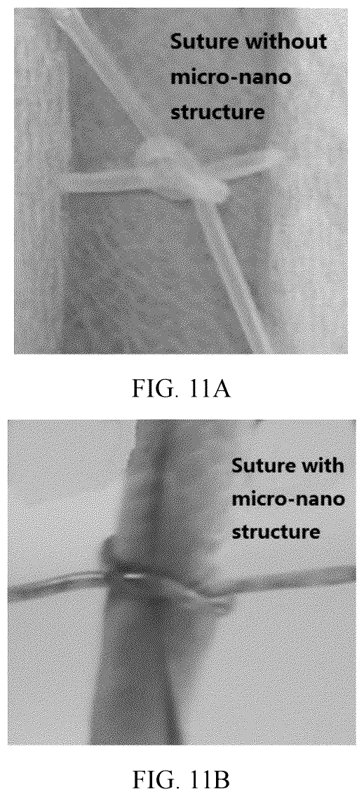

Referring to FIGS. 11A and 11B, a schematic diagram of the knotting of the bioelectronic surgical suture with or without surface micro-nano structures, which is involved in the embodiments described in the present disclosure, is described in detail. When using a bioelectronic surgical suture without surface micro-nano structures, the outer wall of the capillary is smooth, the friction effect is weak, and it is easy to loosen when knotting; when there are micro-nano structures on the outer wall (i.e., using a bioelectronic surgical suture with surface micro-nano structures,), the roughness of the wall surface increases and the friction force increases, which can more effectively knot the surgical suture on the same test skin.

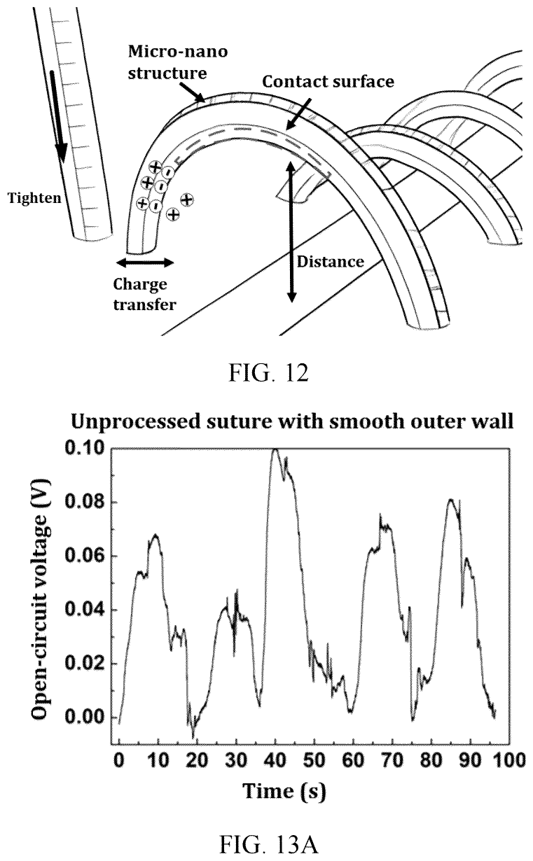

Referring to FIG. 12, the principle of monitoring suture tension of bioelectronic surgical suture involved in the embodiments described in the present disclosure is described in detail. The distance between the contact surface of the bioelectronic surgical suture (i.e., the surface where the surgical suture contacts the sutured tissue) and the sutured tissue is shortened under the change of tension. Based on the principle of triboelectric nanogenerator, the difference in the ability to gain and lose electrons between materials causes charge transfer, generates current, and outputs electrical signals. This effect produces signals with different voltage amplitudes. By analyzing the amplitudes of these signals, the force exerted on the surgical suture can be quantified, achieving dynamic monitoring of the suturing force.

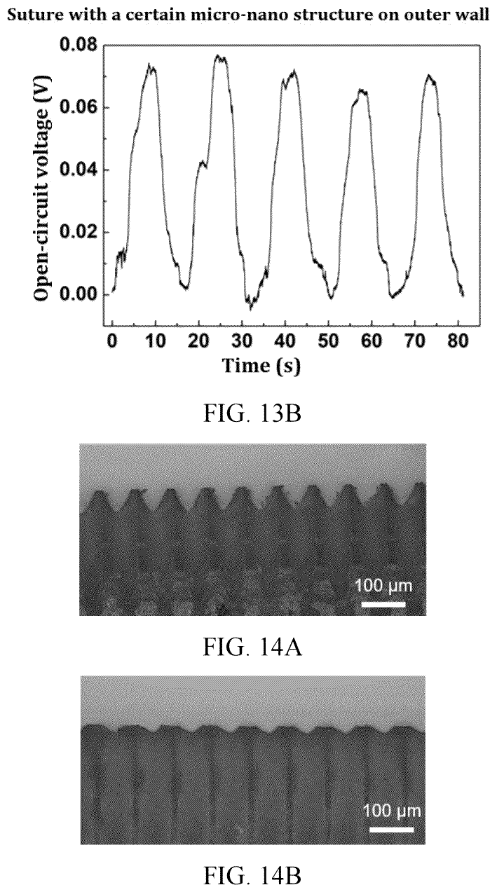

Referring to FIGS. 13A and 13B, a comparison diagram of the signals generated by the contact between the bioelectronic surgical suture with or without surface micro-nano structures and the sutured tissue in the embodiments described in the present disclosure is described in detail. When using a bioelectronic surgical suture without surface micro-nano structures, the signal generated by the contact between the bioelectronic surgical suture and the sutured tissue is irregular, and there are large interfering signals and burrs, and the sensitivity is only about 0.04 V/N. With a certain micro-nano structure, the signals generated by the contact between the surgical suture and the sutured tissue are more regular, and the signals output cyclically under the same force can maintain good consistency with almost no interference. This comparison further demonstrates the importance of surface micro-nano structures for achieving stable and distinct output signals.

Note that this part of the comparison only includes the initial comparison of a bioelectronic surgical suture without surface micro-nano structures, and the monitoring performance can be further improved by optimizing the micro-nano structure parameters.

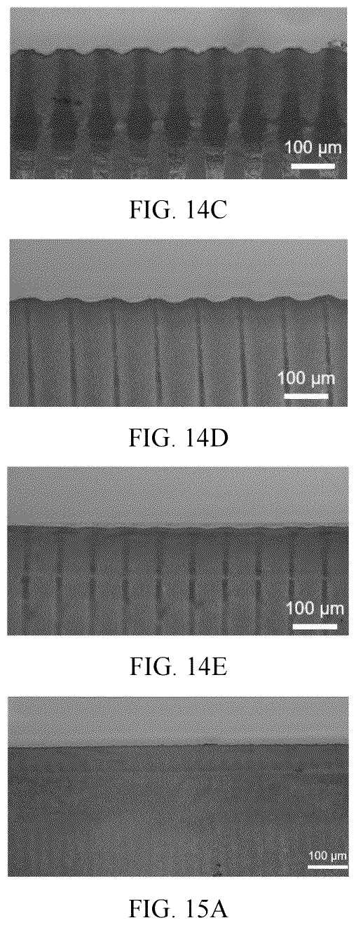

Referring to FIGS. 14A-14E, a schematic diagram of the surface micro-nano structure is shown, where the speed of the femtosecond laser is changed to change the depth of the micro-nano structures.

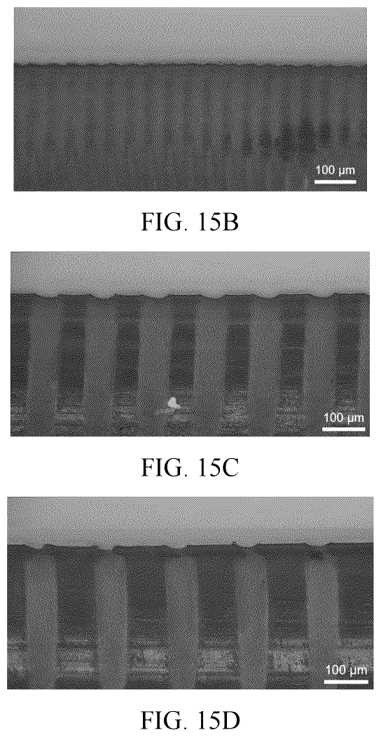

Referring to FIGS. 15A-15D, a schematic diagram of the surface micro-nano structure is shown, where the line spacing of the femtosecond laser is changed to change the spacing of the micro-nano structures.

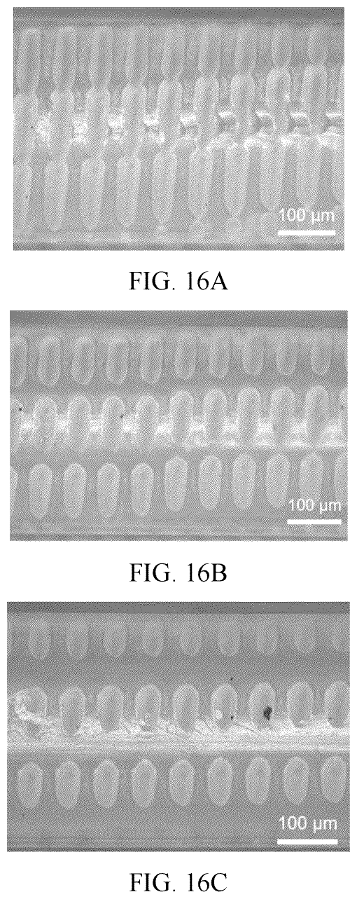

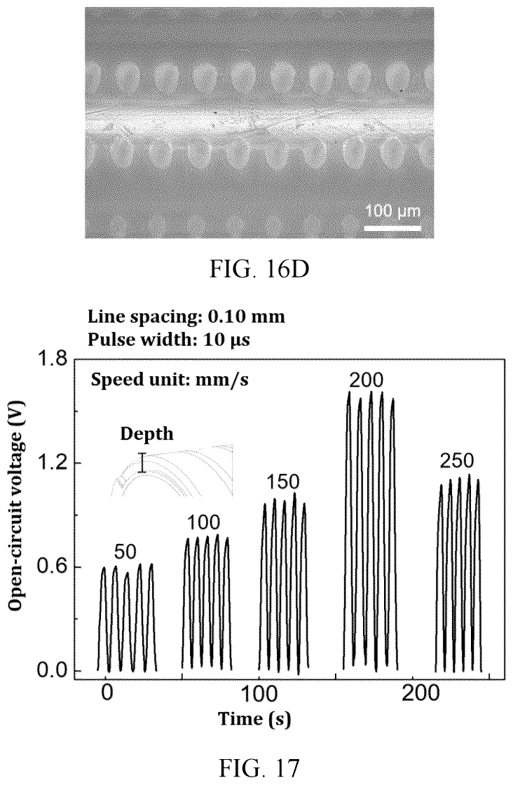

Referring to FIGS. 16A-16D, a schematic diagram of the surface micro-nano structure is shown, where the pulse width of the femtosecond laser is changed to change the continuity of the micro-nano structures.

Referring to FIG. 17, comparing the output signals generated by the contact between the bioelectronic surgical suture and sutured tissue involved in the present disclosure under different speed parameters of femtosecond laser, the change in the depth of the micro-nano structures affected the contact area between the surgical suture and sutured tissue. Both too deep and too shallow depths reduced the contact area, and it was concluded that the laser speed of 200 mm/s is optimal.

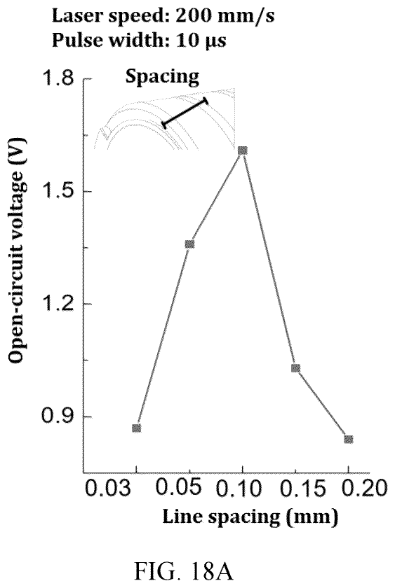

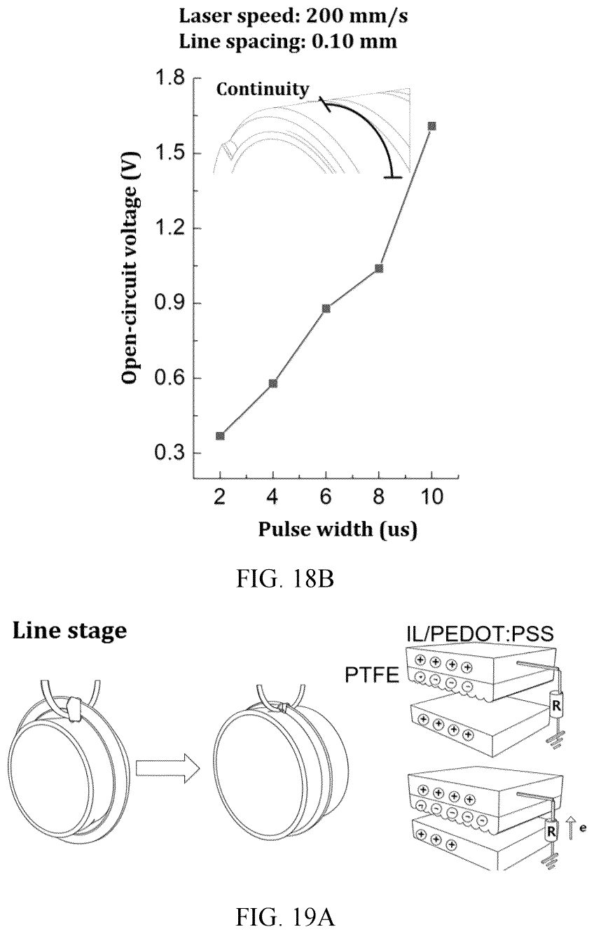

Referring to FIGS. 18A and 18B, comparing the output signals generated by the contact between the bioelectronic surgical suture and sutured tissue involved in the present disclosure under different line spacing parameters and pulse width parameters of femtosecond laser, the change in the spacing and continuity of the micro-nano structures affected the contact area between the surgical suture and sutured tissue. It was concluded that the optimal laser line spacing is 0.1 mm and the optimal pulse width is 10 μs.

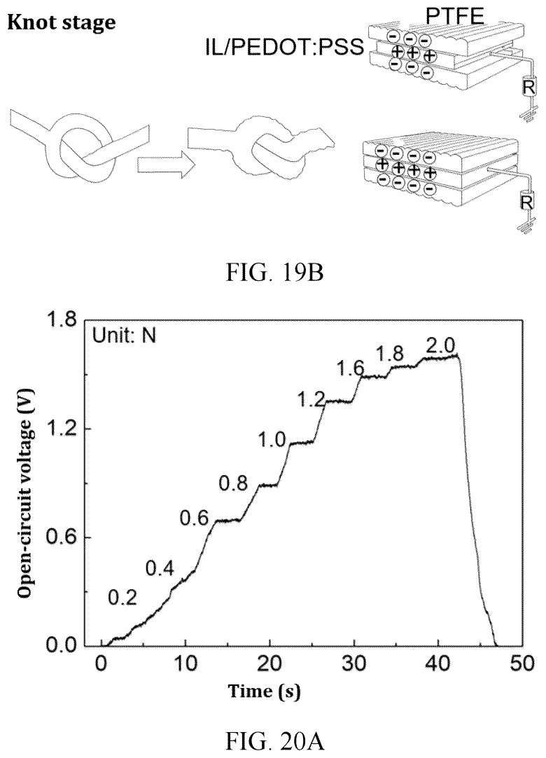

Referring to FIGS. 19A and 19B, the friction stage between the bioelectronic surgical suture and the sutured tissue involved in the embodiments described in the present disclosure is described in detail. The suture process can be divided into a line stage and a knot stage. The surface of the bioelectronic surgical suture involved in the line stage first came into contact with the tissue. During this process, a triboelectric effect occurred between the bioelectronic surgical suture and the tissue, resulting in voltage changes. Once the contact between the bioelectronic surgical suture and tissue reached a predetermined threshold, tightening of the knot played a leading role in the suture. The knot of the bioelectronic surgical suture continued to tighten under external force, while the inner conductive material displaced relatively between the inner walls.

For the bioelectronic surgical suture, a comparative test of simulated suturing force of experimental skin and a comparative test of living biological suture were conducted. For the comparative test of the simulated suturing force of the experimental skin, a tensile testing machine with a force sensor was used to simulate the tension force of the bioelectronic surgical suture on the experimental skin, and a signal acquisition system was used to collect signals. For the comparative test of living biological suture, the bioelectronic surgical suture was used to perform suture comparison on the abdomen of a living rabbit, and a signal acquisition system was used to collect signals. The signal acquisition system includes a signal acquisition card and an electrometer, in which the wire of the bioelectronic surgical suture is connected to the signal acquisition card.

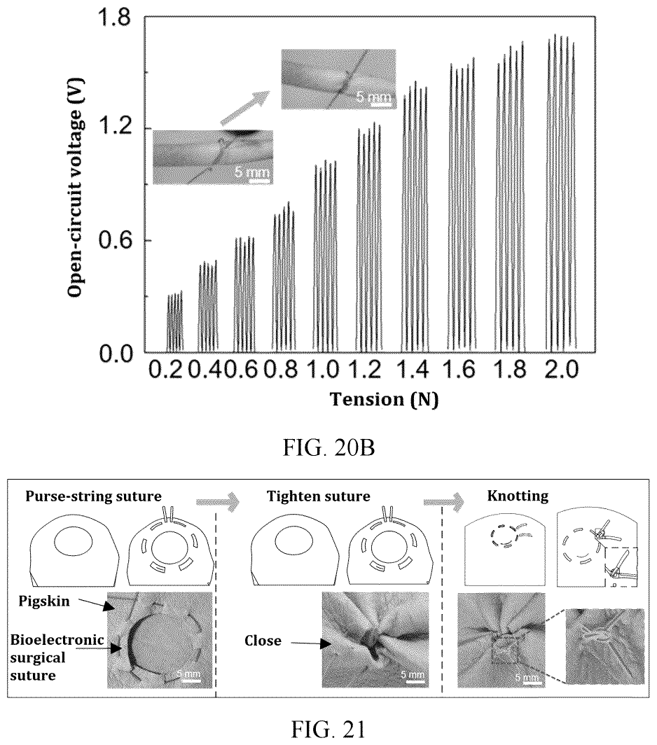

Referring to FIGS. 20A and 20B, comparing the signal changes output by the bioelectronic surgical suture in the present disclosure with the change of tension, an intermittent suture was simulated, and the bioelectronic surgical suture was tied on the experimental skin and knotted. It was concluded that the bioelectronic surgical suture in the present disclosure can well monitor the tension forces of 0-2 N, and the sensitivity of the bioelectronic surgical suture is about 0.95 V/N.

Referring to FIG. 21, the suturing steps of the bioelectronic surgical suture in the present disclosure by simulating purse-string suture method are described in detail:
In step 1: the bioelectronic surgical suture involved in the present disclosure is passed through one circle on the simulated circular wound on the experimental skin according to the shape.
In step 2: the bioelectronic surgery suture is tightened to close the wound.
In step 3: the tightened bioelectronic surgical suture is knotted.

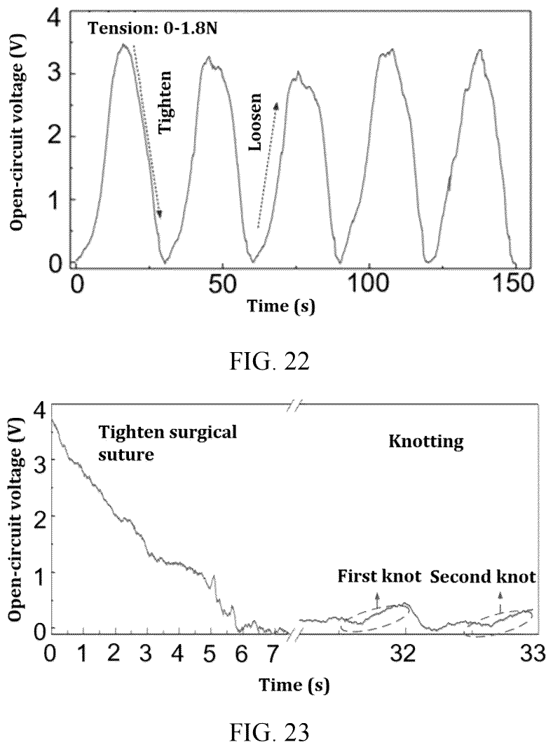

Referring to FIG. 22, the signals that change with the change of tension are compared under the purse-string suture method. The bioelectronic surgical suture involved in the present disclosure is tightened and loosened on the wound, and the signal changes are monitored during the process. It is concluded that the bioelectronic surgical suture involved in the present disclosure can also perform good monitoring under different suture methods.

Note that the actual area of the simulated wound involved in purse-string suture in this part is larger than that involved in FIG. 20, so the initial signal amplitude in this part is larger than that involved in FIG. 20. The ratio of the two initial signal actual areas is close to 1:2, so under the same range of tension, the ratio of the output signal involved FIG. 20 to the output signal involved in this purse-string suture experiment is also close to 1:2.

FIG. 23 is a schematic diagram of real-time monitoring signals during the entire stage of purse-string suture.

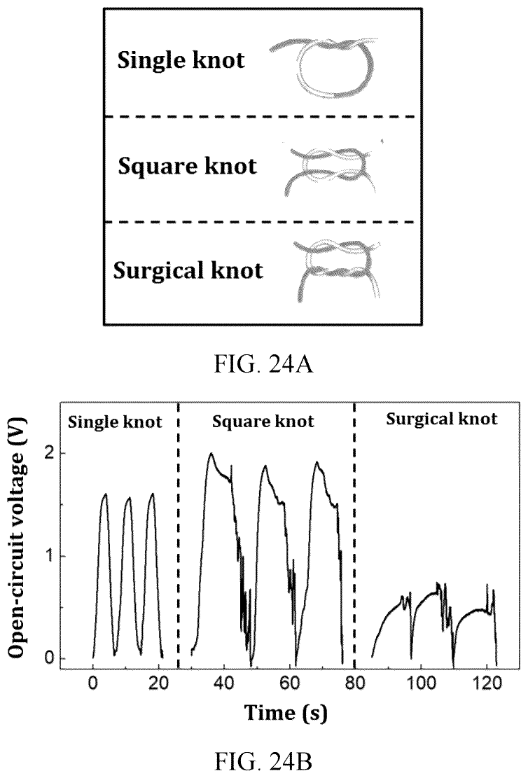

FIGS. 24A and 24B are schematic diagrams of signal changes in different knotting methods of the bioelectronic surgical suture involved in the present disclosure. The knotting methods involve commonly used clinical knotting methods: single knot, square knot and surgical knot.

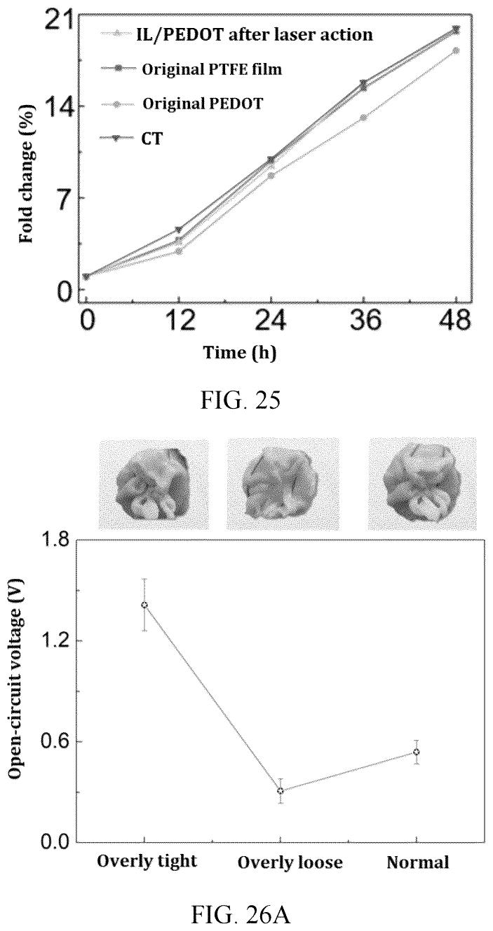

FIG. 25 is a schematic diagram of the biocompatibility results of materials involved in embodiments of the present disclosure.

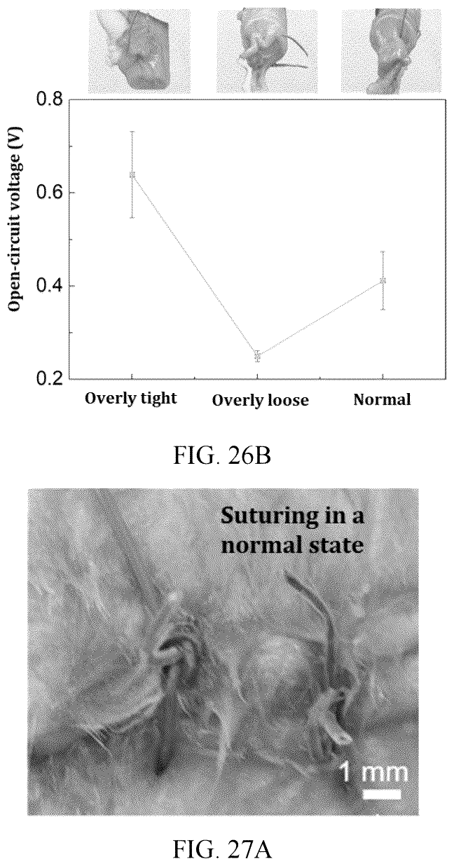

FIGS. 26A and 26B show that the bioelectronic surgical suture in the present disclosure uses purse-string suture to suture isolated biological large intestine and small intestine tissues in three different suture states (normal, overly loose, and overly tight). The output signal changes were compared with the measured tension changes.

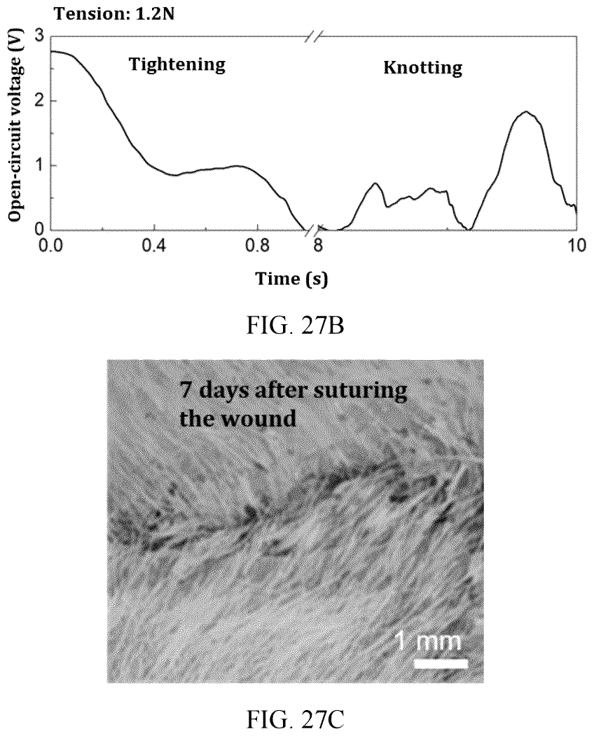

FIGS. 27-27D are schematic diagrams of the bioelectronic surgical suture involved in the present disclosure suturing the abdomen of a living rabbit in a normal state. The real-time output signal changes are compared with the measured tension changes. The tissue healing conditions with different suture effects after 7 days compared, and it is concluded that the bioelectronic surgical suture involved in the present disclosure can be effectively used to monitor the suture state without adversely affecting conventional healing.

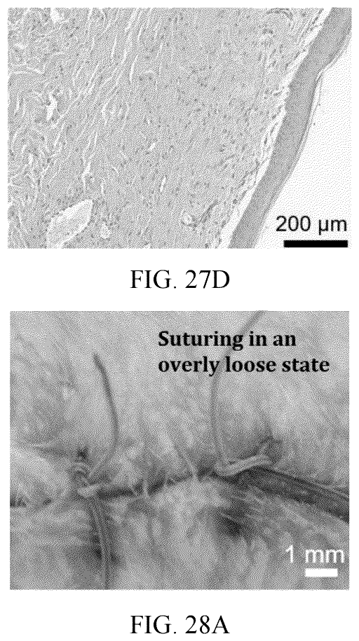

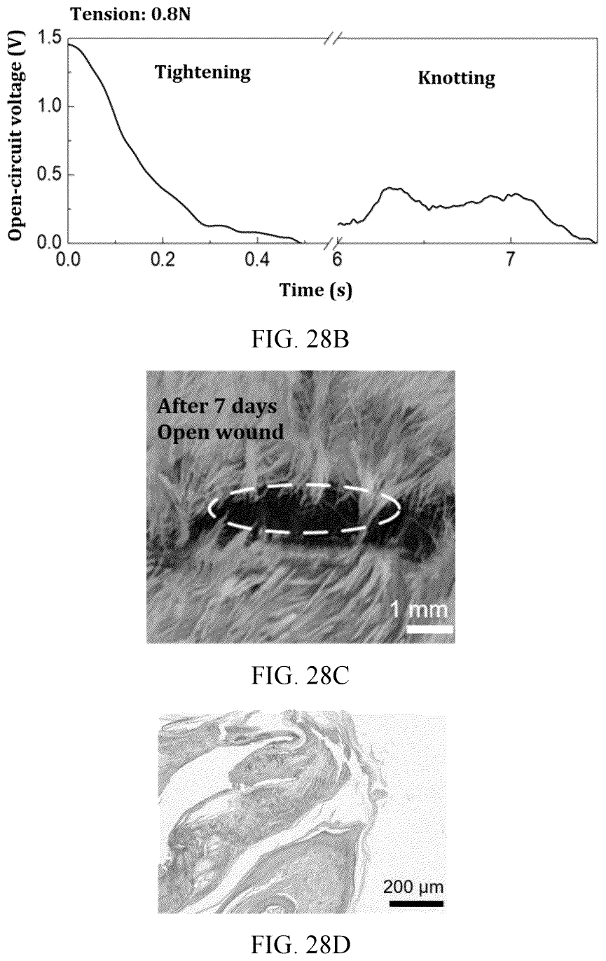

FIGS. 28A-28D are schematic diagrams of the bioelectronic surgical suture involved in the present disclosure suturing the abdomen of a living rabbit in an overly loose state. The real-time output signal changes are compared with the measured tension changes. The tissue healing conditions with different suture effects after 7 days compared, and it is concluded that the bioelectronic surgical suture involved in the present disclosure can be effectively used to monitor the suture state without adversely affecting conventional healing.

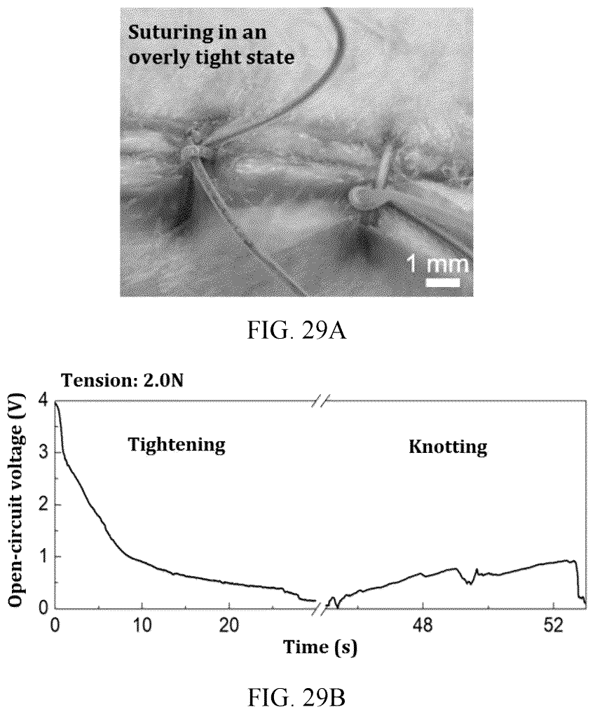

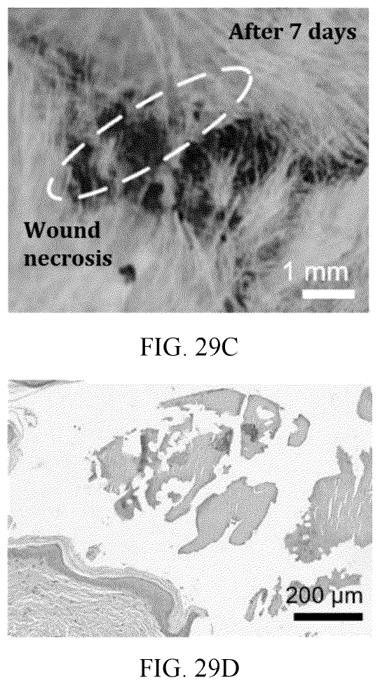

FIGS. 29A-29D are schematic diagrams of the bioelectronic surgical suture involved in the present disclosure suturing the abdomen of a living rabbit in an overly tight state. The real-time output signal changes are compared with the measured tension changes. The tissue healing conditions with different suture effects after 7 days are compared.

In summary, it is concluded that the bioelectronic surgical suture involved in the present disclosure can be effectively used to monitor the suture state without adversely affecting conventional healing.

In embodiments of the present disclosure, the sutured tissues that can be monitored by the bioelectronic surgical suture include but are not limited to biological epidermal tissues and internal tissues.

In the embodiments of the present disclosure, the suture methods that can be monitored by the bioelectronic surgical suture include but are not limited to intermittent suture, continuous suture, purse-string suture, etc.

In embodiments of the present disclosure, the knotting methods include but are not limited to single knot, square knot, surgical knot, etc.

In the description of this specification, a person of ordinary skill in the art can combine and integrate the embodiments or examples described in this specification and the features of the embodiments or examples unless they are in conflict with each other.

The above are only preferred embodiments of the present disclosure and are not used to limit the present disclosure. Any modifications, equivalent substitutions, simple improvements, etc., made to the substantive content of the present disclosure are to be included within the scope of protection of the present disclosure.

1. A bioelectronic surgical suture for monitoring suture tension, comprising:
a conductive material made of electropositive material, and
a sleeve made of electronegative material, wherein
the sleeve is a hollow capillary, the conductive material is arranged in the sleeve through infusion, and is processed by a first laser scanning operation after infusion; an outer wall of the sleeve has a micro-nano structure manufactured by a second laser scanning operation; the bioelectronic surgical suture is a self-powered sensor based on a principle of triboelectric nanogenerator; and the suture tension of the bioelectronic surgical suture causes changes in a contact area between the bioelectronic surgical suture and a sutured tissue, and output electrical signals changes to monitor the suture tension on the bioelectronic surgical suture.

2. The bioelectronic surgical suture of claim 1, wherein the conductive material is a biocompatible conductive polymer selected from at least one of conductive hydrogels, poly (3,4-ethylenedioxythiophene)-polystyrene sulfonate (PEDOT:PSS), ionic liquid mixed PEDOT:PSS (IL/PEDOT:PSS), carbon materials, or metallic materials.

3. The bioelectronic surgical suture of claim 1, wherein the sleeve is a biocompatible polymer selected from at least one of polytetrafluoroethylene (PTFE), polydimethylsiloxane (PDMS), silicone, or polyurethane (TPU).

4. The bioelectronic surgical suture of claim 1, wherein an outer diameter size of the sleeve corresponds to an existing surgical suture used in clinical applications, and two ends of the bioelectronic surgical suture are arranged with a suture needle and a wire; and the suture needle is connected to the sleeve for penetrating the sutured tissue, and the wire is connected to the conductive material for connecting signal acquisition equipment.

5. The bioelectronic surgical suture of claim 1, wherein the first laser scanning operation comprises: scanning the conductive material along the sleeve with a first laser, rotating the sleeve to change an angle of the sleeve, and scanning the conductive material again; wherein the first laser is a continuous-wave laser (CW laser) with a wavelength of 532 nm, a laser power control range of 0.02-0.1 W, a speed control range of 50-200 mm/s, and a line spacing control range of 0.01-0.05 mm.

6. The bioelectronic surgical suture of claim 1, wherein the second laser scanning operation comprises: scanning the conductive material along the sleeve with a second laser, rotating the sleeve to change an angle of the sleeve, and scanning the conductive material again; wherein, the second laser is a carbon dioxide infrared laser, a femtosecond infrared laser, or a femtosecond ultraviolet laser, with the second laser being controlled at a frequency of 50-100 KHz, a speed of 50 -250 mm/s, a line spacing of 0.03-0.2 mm, and a pulse width of 2-10 μs to control the effect of the second laser's thermal impact.

7. The bioelectronic surgical suture of claim 1, wherein the bioelectronic surgical suture is capable of providing different quantitative feedback according to different suture methods, different suture sites, or different knotting methods.
